(19) United States
(12) Patent Application Publication
(10) Pub. No.: US 2017/0209346 A1
(43) Pub. Date: Jul. 27, 2017

(54) SHAPED CELLULOSE BODIES WITH PHYSIOLOGICALLY ACTIVE MINERAL SUBSTANCES DISTRIBUTED THEREIN

(71) Applicant: smartpolymer GmbH, Rudolstadt (DE)

(72) Inventors: Marcus KRIEG, Weimar (DE); Ralf-Uwe BAUER, Rudolstadt (DE); Michael MOOZ, Saalfelder Höhe (DE); Sabine RIEDE, Ulhst?dt-Kirchhasel (DE)

(21) Appl. No.: 15/301,637

(22) PCT Filed: Apr. 8, 2015
(86) PCT No.: PCT/EP2015/057629

§ 371 Date: Oct. 3, 2016

(30) Foreign Application Priority Data
Apr. 8, 2014 (DE) 10 2014 104 965.9

(51) Int. Cl. A61K 8/02; A61Q 19/00; A61K 8/19; D01F 2/00; D01F 1/10

(52) U.S. Cl. CPC A61K 8/0204; D01F 2/00; D01F 1/10; A61K 8/19; A61Q 19/00; D10B 2509/00; A61K 2800/74; A61K 2800/56

(57) ABSTRACT

What is described is a shaped cellulose body having physiologically active mineral substance compounds distributed therein, which are bound within the cellulose matrix of the shaped body and are distributed homogeneously over the cross section thereof. The mineral substance compound is soluble in water, and contains at least one element selected from the group comprising sodium, potassium, magnesium, calcium, iron, copper, manganese and zinc. Even after repeated washing, the shaped cellulose body still contains a high proportion of the mineral substance. It preferably takes the form of fibers, filaments, films or spunbonded webs formed from these fibers. It is especially intended for topical purposes on the human skin, specifically for cosmetic or dermatological purposes. These shaped bodies can be processed to give sheetlike structures, laminates, composite materials and webs, alone or in a mixture with other shaped bodies and fibers.

The invention relates to cellulosic functional moldings incorporating water-soluble mineral compounds in the cellulose matrix throughout the entire cross section of the molding. The moldings are capable of releasing minerals for a prolonged period. They are suitable for multiple use and durable to washing.

Minerals, also known as trace elements, have a positive effect on the human body by balancing the water and electrolyte regime and acting as cofactors to many enzymes and so participating directly in the metabolism. Important cofactors of metabolic processes are the minerals sodium, potassium, magnesium, calcium, selenium and zinc.

Cellulosic fibers useful as carriers for active ingredients are known. Usually, as in DE19911041, the cellulosic fibers form part of cosmetic preparations and are impregnated with active cosmetic and dermatological ingredients. They have the disadvantage that they are only suitable for a single use and do not meet the requirements of a multi-trip product. A further disadvantage of DE19911041 is that the active ingredients are nanoparticles which first have to be burdensomely formed and prepared. Moreover, the use of nanoparticles and their effects on health and the environment are a matter of controversy, so their use is more and more eschewed.

DE 44 26 966 discloses cellulosic fibers containing solid additives. These are particularly pigments or fillers notable for durable incorporation in the fiber. Metal oxides are inter alia also mentioned. These additives do explicitly meet the objective of a very durable incorporation and are suitable for a no-release multi-trip product. These fibers accordingly only contain the metal oxides in a state of poor bioavailability.

WO2010025858 discloses a cellulosic molding having a high degree of whiteness and antibacterial properties, which contains zinc-containing pigments and still has an antibacterial effect even after 50 washes. There is nothing here about a mineral being delivered, the objective being a long-term antibacterial effect and a high degree of whiteness.

WO2009062657 discloses cellulosic moldings having a cellulose matrix incorporating therein dispersed inclusions of apolar organic compounds, although these inclusions may also be fat-soluble actives, such as vegetable oils or fat-soluble vitamins. Water-soluble actives cannot be incorporated in this way.

EP1633375 describes a composition for long-term delivery of Mg for use to cosmetic or therapeutic purposes in nutrition. MgCl₂ is the main source of magnesium. MgCl₂ is a readily water-soluble compound and therefore an envelopment with a biologically dissoluble film is required to ensure the long-term delivery.

DE202010010803 describes a fabric comprising fibers from eucalyptus wood pulp (e.g., lyocell fibers from said pulp) and elastane, admixed with a flexible proportion of ZnO and suitable for garments, particularly underwear. The eucalyptus wood yarn comprises about 80% of eucalyptus wood fiber and about 20% of ZnO-containing fiber. There is no mention as to whether the ZnO fiber has a ZnO coating, or whether the ZnO is dispersed in the fiber or whether a cellulosic fiber is concerned.

CN 101230495 describes a method of forming cellulosic fiber when pulp is dissolved in an ionic liquid and the resulting solution is mixed with tourmaline powder having a particle size of 10 to 400 nm. The spinning solution obtained is spun into a coagulation bath. The coagulated fiber is subsequently drawn, washed and dried. The presence of tourmaline powder confers special electrical properties on the fiber. The fiber releases anions on exposure to an external agency (heat, pressure/impact) and acts bacteriostatically and fungistaticaily. The fiber is particularly intended for apparel fabrics and textiles in the medical sector. Particle sizes used in the method of CN 101230495 must be classified as belonging to the domain of nanoparticles.

In addition, a fiber is known that contains algal powder and is marketed under the brand name Seacell™. Yet the mineral content of algal powder is relatively low at between 0.3-13% of the dry powder, depending on the algal species. In addition, algae have ion exchanger properties and hence an ability to bind metals and a reluctance to release them.

WO2009036481 discloses a lyocell fiber containing 0.07-5 wt %, based on the fiber, of pearl powder. CaCO₃ is the main constituent of pearl powder/mother of pearl. This pearl powder, being integrated in an organic matrix, is by its very nature water-insoluble. Pearl powder is accordingly unsuitable for releasing/delivering minerals under the conditions present on the human skin.

The problem addressed by the present invention was therefore that of providing a cellulosic molding having textile characteristics which contains physiologically, particularly dermatologically, active mineral compounds and is capable of releasing mineral ions when applied to the skin. The carrier/molding shall be suitable for multi-trip use. The release of mineral ions or of further active ingredients on the skin shall take place at a very even rate across not less than 5 to 100, preferably up to 10 to 50 use cycles. One use cycle in a textile application, for example a garment worn next to the body, is made up of the period of using the textile plus the domestic wash which then usually follows. The end of this domestic wash marks the start of the next use cycle for the textile.

The following were to play no part in solving the problem:
Nanoparticles, since these are burdensome and hence costly in manufacture and use and are rejected by the consumer because of possible adverse consequences for health and the environment.
Inorganic pigments and materials in non-hydrosoluble, non-bioavailable form.
Components of variable composition such as vegetable and animal products. The quality and chemical composition of such additives is inconsistent, varying from batch to batch of the raw material. Moreover, the mineral content of these products is often so low that to achieve the delivery/release effect desired, very large amounts of these products would have to be incorporated in the moldings. The requisite amounts of often more than 30 wt % additive impose severe constraints on the technical possibilities for manufacturing such moldings. The physical properties of such high-filled moldings versus unfilled or low-filled moldings evince low values for mechanical strengths, which severely limits processing and use.
Additional ion exchangers as a component. This additive makes the material costly, since a burdensome grinding process is needed for the ion exchange resin and the carrier material having the ion exchanger function first has to be produced in an initial operation. After the carrier material has been obtained, it often has to be converted from the as-obtained sodium form into the desired form by ion exchange in a further operation. A burdensome multi-step manufacturing process is accordingly required.
Additional envelopments/encapsulations for the active ingredients to ensure long-term activity.

The problem is surprisingly solved by cellulosic moldings incorporating inorganic mineral compounds forming a homogeneous distribution throughout the cross section of the cellulose matrix that are characterized in that the mineral compounds bind in the cellulosic molding and contain and can release at least one water-soluble mineral, preferably at least one cosmetically and/or dermatologically active mineral. The mineral compounds contain at least one mineral from the group consisting of sodium, potassium, calcium and magnesium and zinc. Mineral compounds containing at least one mineral from the group consisting of calcium, zinc and magnesium are particularly preferable. Water solubility is a prerequisite for the activity of the mineral compounds used, since corresponding anions and cations can thus be released. Mineral compounds deemed water-soluble for the purposes of this invention have a solubility of 0.5 to 2000 mg/l, preferably of 1.0 to 100 mg/l, all in distilled water at 20° C. and a neutral pH.

The solubility in water preferably increases in a slightly acidic milieu. The skin surface of a human being has such a slightly acidic milieu. The tricky bit was for the as-obtained final molding still to contain water-soluble mineral compounds in a sufficient amount to ensure any dermatological activity. Their production process requires lyocell moldings to be washed very thoroughly and with plenty of water in order that no solvent residues remain in the molding. The constant supply of fresh water makes it impossible for a solution equilibrium to become established for the mineral ions. The as-extruded (as-spun) moldings have to have a high mineral content in order that the final product still contains a dermatologically effective level of mineral ions following the washing and aftertreating operation. This is only accomplished by using the lyocell process and establishing a high pH (preferably pH ≧10).

In order that this objective may be achieved, the starting substances added have to contain ≧75 wt % of chemically pure mineral compound based on the overall weight of the starting substance. To ensure a dermatologically or cosmetically effective performance of the minerals across a prolonged period, about 50-100 domestic washes, the level of minerals present in the water-soluble mineral compounds in the final molding has to be more than 2 wt %, preferably more than 5 wt %, based on the weight of the molding.

The term minerals refers to the chemically pure elements and/or ions thereof. Mineral compounds within the meaning of this invention are compounds of these minerals such as oxides, hydroxides, oxyhydrates, salts or complexed compounds.

Particularly preferred mineral compounds are magnesium oxide, magnesium hydroxide, magnesium carbonate, calcium oxide, calcium sulfate, calcium phosphate, calcium hydrogenphosphate, hydroxylapatite, zinc oxide, zinc carbonate, and also naturally occurring minerals which contain sodium, potassium, calcium and/or magnesium in high weight fractions in water-soluble form.

The mineral compounds are used in unencapsulated, unsupported form during the production of the molding. The mineral compounds are preferably in a chemically pure form in order to avoid adverse side-effects or impurities and to better police the effect.

The incorporation in a cellulose fiber of mineral compounds having a solubility of 0.5 to 2000 mg/l in water has the considerable disadvantage that a significant proportion of these compounds will pass into the aqueous spin- and washbaths even as the fiber is being formed. These mineral compounds and released minerals are accordingly unable to contribute to the intended dermatological and cosmetic effect of the fiber, having already been lost via aqueous baths at the production stage. The contamination of the spin- and washbaths with such mineral compounds also disrupts the fiber-forming process. The acidic conditions of the viscose process will leach out particularly many mineral compounds and/or minerals. The rate of release is likewise severe under the typical conditions of the lyocell process, typically involving a spin- or washbath pH between 7 and 8. Prior art solutions accordingly make use of auxiliary materials, examples being natural or artificial ion exchange materials, in order to bind the incipiently dissolved minerals in the fiber, and/or the fibers are coated with these auxiliary materials. The disadvantages of these auxiliary materials have already been explained.

In order to secure the intended cosmetic and dermatological effect of the cellulosic moldings according to the present invention, the molding process must not release the minerals to any significant degree, if at all.

A solution to this problem was achieved by incorporating the inorganic particles such as mineral compounds at hitherto uncustomary highly elevated pH in the aqueous spin- and washbaths.

Surprisingly, the release of relevant minerals in spin- and washbaths is distinctly reduced by raising the spin- and washbath pH in the lyocell process to an untypical value of above pH 10. An increase in the pH is readily achievable by the addition of NaOH or of other hydroxides into the spin- and washbaths. Particularly NaOH is already being used in the lyocell process, in small amounts, for the purpose of pH control and therefore is not an additional process chemical. NaOH is efficiently removable from the aqueous NMbMO baths by the customary desalting practiced in the course of the regeneration of the spin- and washbaths which is customary in the lyocell process, and is recyclable.

This solution was unforeseeable because a person skilled in the art knows that cellulosic fibers swell substantially at above pH 10. The cellulose structure opens as well as swells and thus improves accessibility for materials incorporated into the cellulose matrix. An increased loss of minerals would thus have been predicted. A known countervailing process is the solubility of mineral compounds decreasing with increasing pH. The two competing processes in the use described herein, however, unexpectedly lead to a substantial reduction in the solubility of the mineral compounds in the production process of the fiber and hence to a stabilization of the inorganic particles and mineral compounds incorporated into the fiber. As a result, these particles transfer almost completely into the final cellulosic fiber and are available for the intended cosmetic or dermatological applications of products fabricated therefrom.

The mineral compounds are usually incorporated in powder form. The powders have a wide particle size distribution. The average diameter is generally in the range from 0.5 to 100 μm, preferably in the range from 0.5 to 20 μm. Where the particles are not spherical but differently shaped (e.g., needle-shaped or platelet-shaped), the average diameter is to be understood as meaning the average equivalent sphere diameter. The particle size may be determined by light diffraction, for example with instruments from Sympatec which use laser light diffraction.

The maximum particle size (equivalent sphere diameter) shall not be more than 200 pun, particularly not more than 50 μm. When the cellulosic moldings are fibers, the average diameter of particles will be distinctly smaller than the diameter of the fibers, so as not to impair the mechanical stability of the fibers. What will prove advantageous in this case is an average diameter (equivalent sphere diameter) of 0.5 to 10 μm, in particular from 1 to 5 μm, and a maximum diameter of 20, in particular of 10 μm.

The cellulosic moldings of the present invention preferably take the form of filaments, fibers, self-supporting films/sheets or fibrous nonwoven webs.

However, it is also possible for the particle powders to be incipiently dissolved in water and introduced in that form into the spinning solution during the production of the cellulose solution. Dissolving cellulose in the preferred lyocell process requires the removal of excess water under reduced pressure. Interactions of the solvent with the hydrogen bond system of the cellulose creates a spinning solution which is moldable. At the same time, the incipiently dissolved mineral compounds are converted back into an insoluble form by a shift of the dissolution equilibria in the cellulose/solvent/water/mineral compound(s) system. This dissolving and reprecipitating causes the mineral compounds to be built into the cellulose scaffold in finer and more uniform state of subdivision, and to dissolve less rapidly out of the final cellulosic molding. In the case of cellulosic fibers, this simultaneously serves to reduce the surface roughness of the cellulosic fiber obtained. A low surface roughness is important for any processing of the fibers into yarns and textile structures.

One embodiment of the invention provides that inorganic compounds and mineral compounds having differing solubility in water be used.

The combination of appropriate mineral compounds differing in water solubility serves to promote the release for a longer period and to influence the solubility of the components with respect to each other. Different mineral compounds having different solubilities in water are accordingly incorporated. This ensures a long-lasting period of release. The first ingredient to be released is the mineral compound which has the greatest solubility but which at the same time suppresses the solubility of the other compounds. It is only when this readily soluble mineral compound is all used up that the compound having the lower solubility becomes active. This provides for the release of a significant amount of ions for a long period. A person skilled in the art knows that the admixture of a more soluble magnesium salt to an aqueous mixture of a less soluble magnesium salt will further suppress the solubility of the latter. This effect is unaltered for compounds in the interior of lyocell fibers and can thus be utilized for the controlled release of minerals. The same applies to further active ingredients included in the interior of the mineral compounds or sorbed thereon. In a mixture of two or more mineral compounds, at least one thereof has a water solubility in the range from 0.5 to 2000 mg/l, preferably from 1.0 to 100 mg/l, the water solubility of this compound being at least three times higher or lower than that of the other and/or at least one other.

How fast and to what extent minerals are released is determinable by using suitable methods such as ICP-MS or ICP-OES to measure the minerals in eluates. This comprises determining the minerals in aqueous, oily or solvent-containing eluates or determining the minerals in materials that were contacted with the claimed molding in any form. It is for instance possible to place a textile fabric comprising the claimed moldings in a defined volume of artificial acidic perspiration (to DIN 54233-3) for a defined period and to determine the accumulation of dissolved minerals. This measurement may be repeated after the textile fabric has been washed a certain number of times. When this is done, the concentration of minerals detectable in the artificial perspiration after 10, preferably 50 washes should still be not less than 10% of the initial concentration. The initial concentration is the measured value obtained for the unwashed textiles.

Differing solubility is selected not just by selecting different mineral compounds but also by selecting different solubilities for an individual compound. Magnesium oxide, for instance, is available in different degrees of sintering, particle sizes or particles of differing surface area, covering the range from readily soluble to sparingly soluble. The selection of suitable magnesium oxides, or mixtures thereof, thus likewise leads to the desired outcome of a bioavailable but sustained release of mineral and active ingredients. The degree of sintering or the particle structure controls the water solubility of metal ions from oxides and salts and thus controls the rate of release. The object, i.e., the controlled and sustained release of minerals, is thus achieved in the case of magnesium, for example, by using MgO at low and middling degrees of sintering in admixture. Incipiently calcined NgO is quick to form hydroxides and hence is rapidly soluble. Extremely highly calcined magnesium oxide is virtually impervious to water and scarcely releases any magnesium ions. Similar effects regarding magnesium release are also attainable by using or combining oxides differing in activity. Different activities are due to different surface areas (as measured, for example, by nitrogen absorption using the method of Brunauer, Emmett and Teller—the BET method) of compounds, which influence the solubility in water.

Sintered yet still water-soluble magnesium oxides, like other usable mineral compounds as well, are also notable for a high porosity. This allows the utilization of the pores for the inclusion, sorption of further actives, such as vitamins, glidants, antioxidants, polyphenols, extracts of natural curative and reconditioning agents such as aloe vera, horse chestnut, etc. The large specific surface area of the mineral compounds also permits sorption to/on the surface. Further cosmetically and/or dermatologically active substances for inclusion in the pores include vegetable oils, essential oils, scents, scent compositions, fatty acid esters, ethers and anhydrides of vegetable, animal or synthetic origin, long-chain alcohols, fatty acids, plant products such as palm oil, jojoba oil, evening primrose oil, avocado oil, plant extracts, paraffins and paraffin mixtures formed from n- or iso-alkanes, fat-soluble vitamins such as vitamins E, A and D, and mixtures thereof. This provides cellulosic moldings of complex dermatological or cosmetic activity. One direction of this activity is that as cofactors in metabolic processes, for example as antiaging agents. It is a cosmetological fact that co-applied lipophilic and hydrophilic substances have a higher rate of penetration [Raab, W., Kindl, U.: Pflegekosmetik, Wissenschaftliche Verlagsgesellschaft Stuttgart (2004) p. 20].

The aforementioned concept of including active ingredients into the porous structure of the mineral compounds used makes possible the binding of further cosmetically and dermatologically relevant compounds. A person skilled in the art will here be aware of a multiplicity of compounds from the field of cosmetics. These compounds are used, for example, to regulate the moisture regime of the skin, or are able to ameliorate adverse skin conditions or skin disorders such as wrinkling or acne. It is known that compounds of minerals such as magnesium, calcium or tin, such as talc, magnesia or ZnO are used a powder base. These powder bases have the purpose to accommodate liquid actives in the powder. It will be appreciated that this principle can be co-opted for the purposes of the present invention.

Magnesium carbonate is a further example of a mineral compound having a porous structure. Notably, it has a less alkaline reaction than CaCo₃ and so is less liable to cause irritation of the skin, its covering power is good and it possesses high absorbency for water and fatty chemistries.

The mineral compounds used will naturally have a layered structure in some instances in addition to the porosity described. This layered structure is utilizable for inclusion of actives in the same way as pores. Layered structures, in minerals, examples being sheet-silicates, can thus likewise function as storage media for actives co-incorporated into the lyocell fiber and co-released on the skin. Advantageously, these are likewise active ingredients having a cosmetic or therapeutic, e.g., a dermatological, effect on the skin or the connective tissue.

Cellulosic moldings form part of the subject matter of the present invention because their swellability in water makes them accessible to aqueous media and thus capable of releasing included minerals and active ingredients. Moldings from synthetic polymers, by contrast, are substantially more hydrophobic, so aqueous media do not reach included minerals. Minerals included in hydrophobic polymers are thus not bioavailable. Synthetic polymers thus severely hinder in particular any transfer of the minerals and active ingredients from the textile onto the skin via moisture and perspiration as transfer medium.

Increased release of minerals on contact with the skin versus a reduced release during production and washing of the molding is achieved through the special combination of mineral compounds with the production process of the cellulosic molding via a modified dissolving process (lyocell process). An alkaline pH prevails throughout the entire lyocell process of production. Salts and compounds of important minerals such as magnesium, calcium and tin tend to form hydroxides, oxides, oxyhydroxides, carbonates and bicarbonates at an alkaline pH. This is partly augmented by sorption of CO₂ from the air. These compounds have a lower solubility than compounds of the same minerals in an aqueous acidic milieu. In an acidic milieu, as usually prevails on the skin or in perspiration, the release of minerals from mineral compounds is improved and accelerated. As a result, the minerals are particularly mobilized and available when worn next to the skin. During cleaning of the textile articles, the washing lye causes pH 10 to be reached. Washing lye is included in most commercial laundry detergents because the establishment of an alkaline pH improves the cleaning of fibers, in particular the cleaning of natural fibers. Under these conditions, as with the manufacture of the fibers, any release of the mineral compounds is retarded. This reduced release under alkaline conditions in the wash of the textile articles confers launderability and hence is in accordance with the present invention.

It is thus the use of lyocell technology for producing the intended molding which makes it possible in the first place to produce the described cellulosic moldings at but a minimal loss of mineral compounds. The solvents used for cellulose in the lyocell process are preferably amine oxides, specifically N-methylmorpholine N-oxide monohydrate (NMMO monohydrate), mixtures of N,N-dimethylacetamide and lithium chloride (DMA/LiCl), or ionic liquids, in particular pyridinium, pyridazinium, pyrimidinium, pyrazinium, imidazolium, pyrazolium, piperidinium, pyrrolidinium, [1,2,3]triazolium, [1,2,4]triazolium, thiazolium, quinolinium and isoquinolinium salts, the anions in these salts preferably being chloride, bromide, iodide, cyanate, thiocyanate, perchlorate, formate, acetate, propionate, maleate, fumarate, oxalate, nitrate, tetrafluoroborate, alkanesulfonate or trifluoroacetate ions. Ionic liquids generally have a melting point of less than 200° C. A special mention must be given to 1-butyl-3-methylimidazolium chloride (BMIMCl, melting point about 60° C.), N-butyl-3-methylpyridinium chloride, 1-ethyl-3-methylimidazolium chloride and l-allyl-3-methylimidazolium chloride.

Ionic liquids may be in a blend with organic solvents, such as tetrahydrofuran, dimethylformamide and/or dimethyl sulfoxide.

Lyocell processes dissolve the cellulose purely physically, without any chemical transformation. The solvents used therefor are therefore referred to as direct solvents.

An extrusion process is a particularly convenient way to mold the cellulosic moldings of the present invention. Thereafter the extruded moldings pass through an aqueous-alkaline precipitant bath and also aqueous washbaths. The washbaths remove the solvent for the cellulose in a virtually complete manner. Lastly, the molding is dried.

Other processes for producing cellulosic fibers, for example the viscose process, which comprises transforming the cellulose into cellulose xanthate, have acidic processing conditions and so are distinctly less suitable for producing cellulosic moldings of this type, since a large proportion of the mineral compounds will have already been dissolved out at the stage of forming the moldings and the amounts remaining in the molding are insufficient to ensure an intended release of minerals over more than five use cycles.

Another aspect exploited is the ability of cellulose's naturally present carboxylate groups to form complexed compounds with the minerals and thereby better control the release. Changing the number of carboxylate groups on the cellulosic molding by chemical derivatization or the admixture of substances comprising carboxylate groups, alginates are an example, provides a further possible way to control the release of the dermatologically relevant minerals described.

The cellulosic moldings described, which are preferably fibers and more preferably textile-processable fibers, are useful for making constructs suitable for skin contact. Textiles, fibrous nonwoven webs or papers are particularly suitable for skin contact. These constructs are obtained by known methods of the textile industry, of the nonwovens industry or the paper industry. This includes, for example, wovens, knits, papers and fibrous nonwoven webs. The invention requires the constructs to be suitable for multiple use, i.e., to be washable or launderable.

The term molding comprehends in this case generally any molding formed from a polymer solution by extrusion, blow molding, spinning or pulling, preferably fibers and filaments, self-supporting films/sheets, nonwovens or foams.

Relevant mineral compounds for the purposes of the present invention are those which release sodium, potassium, calcium, magnesium, cobalt, iron, copper, manganese, vanadium, molybdenum, selenium and/or zinc. Some of these are so-called metallic cofactors for important metabolic processes. Minerals are particularly relevant, such as the dermatological elements calcium, magnesium or tin. Calcium and magnesium are particularly preferable. These minerals are a subgroup of the dermatologically relevant actives and cosmetically relevant actives, i.e., from the group of actives that exert a cosmetic or therapeutic effect on the skin or the connective tissue and with the aid of which other chemistries can exerted this effect (co-factors). A multi-trip product or constructs for multiple use are taken by the present invention to be products which are reusable after a domestic hand or machine wash and continue to release minerals when reused.

Relevant mineral compounds for the purposes of the present invention are inorganic compounds of the minerals, in particular oxides, sulfates, carbonates, bicarbonates, silicates, phosphates, hydrogenphosphates, aluminosilicates, oxyhydroxides, hydroxides, fluorides, iodides or chlorides, which have the abovementioned solubility in water at 20° C.

The required partial solubility at 20° C. in deionized water, i.e., in the neutral pH range, is met for example by the hereinbelow recited mineral compounds. This reported solubility is based on literature data. This list in no way restricts the actual multiplicity of possible mineral compounds having the required properties. Moreover, the solubility actually measured under these conditions is material-dependent and may vary substantially owing to different degrees of sintering, particle sizes, active surface areas or porosities and hence differ from these literature values.

Magnesium hydroxide Mg(OH)₂ solubility in water (18° C.): 9 mg/l
Magnesium carbonate MgCO₃ solubility in water (20° C.): 100 mg/l
Calcium carbonate CaCO₃ solubility in water (20° C.): 14 mg/l
Calcium sulfate CaSO₄ solubility in water (20° C.): 2000 mg/l
Calcium phosphate Ca₃(PO₄)₂ solubility in water (20° C.): 20 mg/l
Calcium hydrogenphosphate CaHPO₄ solubility in water (25° C.): 100 mg/l
Hydroxylapatite Ca₅OH(PO₄)₃ solubility in water (20° C.): 6.57 mg/l
Zinc oxide ZnO solubility in water (29° C.): 1.6 mg/l
Zinc carbonate ZnCO₃ solubility in water (15° C.): 10 mg/l
Magnesium oxide MgO 12 mg/l

The required minimal solubility in water is also achieved by many natural mineral compounds. Therefore, these are also suitable for integration into cellulosic moldings in the manner of the present invention with subsequent release of minerals in the manner of the present invention. A prerequisite is that mineral compounds should contain minerals, such as magnesium or calcium, within the meaning of this invention at not less than 75 wt %, based on the weight of the natural mineral compound, so as not to have to add excessively large amounts of additives to obtain a cosmetically or dermatologically relevant effect without the physical textile and clothing-physiological properties of the moldings being unduly impaired.

A person skilled in the art is aware of appropriate groups of natural mineral compounds of the relevant minerals. These need accordingly not be explicitly recited here. Many of these natural mineral compounds are likewise suitable for the cellulosic molding of the present invention.

The examples which follow illustrate the invention. Percentages are by weight unless otherwise stated or directly apparent from the context.

### EXAMPLE 1

Cellulosic fibers comprising mineral compounds were produced according to the lyocell process by using the solvent N-methylmorpholine N-oxide (NMMO) and admixing pulverulent magnesium-containing mineral compounds to spinning solutions of cellulose. The fibers are characterized in that the mineral compounds form a homogeneous distribution throughout the entire cross section of the fiber.

The chemically pure (assay >97%) type 1 magnesium oxide having a solubility in water of 12.5 mg/l (at 20° C.) is added to the spinning solution as a magnesium-containing mineral compound in a concentration of 16.7%, based on the mass of the dry composite fiber. The magnesium content of the lyocell fiber was analytically determined using ICP-OES following acid digestion. The measured initial magnesium content was 7.5%. The durability of the lyocell composite fiber to washing was tested. To this end, the textile was placed in textile wash-bags and washed in a commercially available domestic washing machine (Miele Softtronic Gala Grande XL W6000) using a “short easy-care” program at 40° C. by admixing a commercially available color-type laundry detergent without optical brighteners. The analytically determined magnesium content after 50 washes was still 0.5%.

The chemically pure (assay >99%) magnesium hydroxide having a solubility in water of 3.3 mg/l (at 20° C.) is added to the spinning solution as a magnesium-containing mineral compound in a concentration of 16.7%, based on the mass of the dry composite fiber. The magnesium content of the lyocell fiber was analytically determined using ICP-OES following acid digestion. The measured initial magnesium content was 6.2%. The durability of the lyocell composite fiber to washing was tested. The analytically determined magnesium content after 50 washes was 3.5%.

The mineral compounds magnesium oxide type 1 and magnesium hydroxide were mixed in a ratio of 3:1 and added to the spinning solution in the same way as the individual mineral compound. The overall mineral compound content of the fiber was 16.6%. The magnesium content of the lyocell fiber was analytically determined using ICP-OES following acid digestion. The measured initial magnesium content was 7.6%. The durability of the lyocell composite fiber to washing was tested. The analytically determined magnesium content after 50 washes was now 1.0%. Hence the magnesium content of the fiber comprising the mixture of magnesium oxide type 1 and magnesium hydroxide is twice as high after 50 washes as for the fiber comprising magnesium oxide type 1.

Combining two mineral compounds having different solubilities in the manner of the present invention achieved precise control over the release behavior of the cosmetically active mineral-compound component. Exchanging 4 of the magnesium quantity delivered by the more soluble component magnesium oxide for a less soluble magnesium hydroxide has scarcely any effect on the amount of magnesium in the as-manufactured state, but achieves a doubling in the level of available magnesium after 50 washes versus the use of purely magnesium oxide.

This secures a sustained cosmetic and dermatological activity to textiles fabricated from this fiber material.

TAB 1
Durability of magnesium-containing mineral
compounds to washing

| | | | Analytically | |
|---|---|---|---|---|
| | Magnesium | Analytically | determined | |
| Mineral | content of | determined | magnesium | |
| compound | fiber as | magnesium | content of | |
| and | reckoned | content of | fiber after | Mineral |
| amount used | ex recipe | fiber | 50 washes | release |
| MgO type 1 | 10% | 7.5% | 0.5% | too high |
| (16.7%) | | | | |
| Mg(OH)₂ (16.7%) | 6.9% | 6.2% | 3.5% | too low |
| Combined MgO | 9.2% | 7.6% | 1.0% | optimal |
| type 1 (12.5%), | | | | |
| Mg(OH)₂ (4.1%) | | | | |

1. A lyocell molding incorporating mineral compounds forming a homogeneous distribution throughout the molding cross section, wherein the mineral compounds forming the homogeneous distribution throughout the cross section of the molding bind in the cellulose matrix of the molding and contain at least one water-soluble inorganic mineral compound having the minerals potassium, magnesium, calcium and/or zinc in a more than 2 wt % weight fraction of the minerals as a proportion of the weight of the molding, and release these continuously across 5 to 100 use cycles.

2. The molding as claimed in claim 1, wherein the mineral compound has a solubility of 0.5 to 2000 mg/l in desalted water at 20° C.

3. The molding as claimed in claim 1, wherein the mineral compound is a natural or synthetic compound.

4. The cellulosic molding as claimed in claim 1, wherein said molding contains a mixture of two or more inorganic mineral compounds having differing solubility in water, wherein at least one thereof is a mineral compound as defined in claim 1 which, by itself, has at least a three or more times higher or lower solubility in water at 20° C. than the others or an other.

5. The molding as claimed in claim 1, wherein the mineral is a cosmetically and/or dermatologically active mineral.

6. The molding as claimed in claim 1, wherein said molding is a filament, a fiber, a self-supporting film/sheet or a spunbonded web.

7. The molding as claimed in claim 1, wherein said molding contains additional actives bound on the surface and/or in the pores or layered structures of the mineral compound.

8. A method of releasing mineral ions to skin for topical purposes comprising wearing the cellulosic molding as claimed in claim 1.

9. A method of making the cellulosic molding as claimed in claim 1, wherein said molding is a fiber or filament.

10. The molding as claimed in claim 1, wherein said minerals are present in an amount of more than 5 wt %.

11. The molding as claimed in claim 2, wherein the mineral compound has a solubility of 1.0 to 100 mg/l in desalted water at 20° C.

12. The molding as claimed in claim 3, wherein the mineral compound is-an oxide, hydroxide, oxyhydroxide, carbonate, bicarbonate, sulfate, silicate, aluminosilicate, phosphate or hydrogenphosphate of the elements magnesium, calcium, or zinc.

13. The molding as claimed in claim 3, wherein the mineral compound is a magnesium oxide, magnesium hydroxide, magnesium carbonate, calcium carbonate, calcium sulfate, calcium phosphate, calcium hydrogenphosphate or hydroxylapatite.

14. The molding as claimed in claim 7, wherein said actives are selected from cosmetically and/or dermatologically active vegetable oils, essential oils, scents, scent compositions, fatty acid esters of vegetable, animal or synthetic origin, long-chain alcohols, plant products, plant extracts, paraffins and paraffin mixtures formed from n- or iso-alkanes, fat-soluble vitamins, and mixtures thereof.

15. The molding as claimed in claim 14, wherein said plant products are selected from palm oil, jojoba oil, evening primrose oil, and avocado oil, and said vitamins are selected from vitamins E, A and D.

16. A method of releasing mineral ions to skin as claimed in claim 8 wherein said topical purpose is a cosmetic or dermatological purpose.

17. A method of making the cellulosic molding as claimed in claim 9, wherein said method further comprises incorporating said fiber or filament into a textile, paper or nonwoven.
